(19) United States
(12) Patent Application Publication
(10) Pub. No.: US 2005/0171549 A1
(43) Pub. Date: Aug. 4, 2005

(54) Apparatus and method for treating the spine

(76) Inventors: Frank H. Boehm JR., Utica, NY (US); Benedetta B. Melnick, Rome, NY (US)

Correspondence Address: DILWORTH & BARRESE, LLP, 333 EARLE OVINGTON BLVD., UNIONDALE, NY, 11553, US

(21) Appl. No.: 11/018,609

(22) Filed: Dec. 20, 2004

Related U.S. Application Data
(60) Provisional application No. 60/531,427, filed on Dec. 18, 2003.

(51) Int. Cl. A61B 17/56

(52) U.S. Cl. 606/84

(57) ABSTRACT

An apparatus and method for stabilizing vertebrae and implanting bone growth material is disclosed. Trocars are set onto the specific pedicles of the vertebrae to be stabilized. A series of dilators are passed over each trocar until a final working channel is placed. The interior dilators are removed, leaving the working channel to guide placement of the pedicle screws. The ends of a connecting rod are set in each screw to stabilize the pedicles. A steerable osteotomy device is used via the working channel to obtain the bone graft. Growth material from the graft is then implanted into the stabilized area.

### CROSS-REFERENCE TO RELATED APPLICATION

This application is based on, and claims priority to, U.S. Provisional Application No. 60/405,261 filed on Dec. 19, 2004, the entire contents of which are hereby incorporated by reference.

### BACKGROUND

1. Field of the Invention

The present invention relates to an instrumentation system, and method for operating the same, used in fusion procedures of the spine. In particular, the invention relates to an instrumentation system for subcutaneously interlocking pedicles of adjacent vertebra to be fused and to a minimally-invasive-posterior-approach method of interlocking the pedicles and implanting bone growth material.

2. Description of the Related Art

Surgical treatment for spine disease usually requires spinal implant instrumentation. One of the most common passes screws passed into the pedicles of the patient's vertebrae, with the screws being secured to a connecting cross piece such as a rod or plate to stabilize multiple spinal levels. There are a number of ways to secure the screws to the rod. In general, they include some form of a nut or bolt secured to the screw head to lock the rod or plate in place.

Although plates were initially favored in the early days of spine surgery, a connecting rod is more common in modern procedures. Typically, the rod fits into a cradle in the screw head and then some mechanism secures the rod into the cradle. This generally involves a tightening nut or bolt.

After stabilization of the vertebrae, and removal of problematic tissue, material conducive to bone growth is injected. This is referred to as a surgical fusion. An environment, or milieu, that leads to a bridging, or bony union, of two or more vertebrae is created in the affected area by implanting a bone graft, usually harvested from the patient's iliac crest.

### SUMMARY OF THE INVENTION

It is, therefore, an object of the current invention to provide a system by which a rod, connecting two or more screws that have been placed into the pedicles of the vertebrae of the spine of the patient, can be secured to the heads of these screws.

It is also an object of the invention to provide for such a system in a manner by which the rods are secured to the screws without the use of a nut, bolt, or other securing device that requires the use of a threaded tract.

It is a further object of the invention to provide a system by which the rod is secured to the head of the screw with the use of a ratcheting system.

It is yet another object of the invention to provide a system by which the heads of the screws are provided with a cap or deployable component which, when deployed, will serve to secure the rod to the head of the screw in a nonreversible fashion.

It is even a further object of the invention to provide a series of pins or piston rods on the deployable component of the screwhead.

It is an even further object of the invention to provide a series of channels into which these pins can be secured thus irreversibly securing the rod to the head of the screw.

It is still another object of the invention that the series of channels into which the pins pass are provided with a sequence of narrowed areas ultimately requiring that the pins be ratcheted into position.

It is yet, still, an even further object of the invention to provide a locking mechanism to reinforce the fashion by which the rod is secured to the head of the screw.

These and other objects of this invention are accomplished by providing a system by which pedicle screws are inserted into the bone, and are equipped with screwheads that are provided with several unique features. Among these, a unique feature is a cap which serves as a deployable component. Several embodiments of this component are considered.

In the preferred embodiment, the head of the primary screw has a cap that is positioned directly above the cradle of the screw. Furthermore, a plurality of pins extend from the undersurface of the cap into channels that are found along the walls of the screwhead. These walls, of course, constitute the sides of the cradle. The channels into which the pins pass, are oriented parallel to the long axis of the shaft of the screw. The pins preferably have a swelling at their leading end. Furthermore, the channels have a series of narrowings within them. The length of the channels is such that when the pins have been completely deployed and are inserted entirely within the channels, the cap is securely fitted to the top of the screw. The pins are deployed by a device that causes them to be passed further into the channels. As the leading end of the pins passes through the series of narrowings, it serves to capture the leading end of the pins in an irreversible fashion. Thus, as the cap and its attendant pins are deployed into the top of the screw, the pins are captured sequentially by the narrowings and the cap becomes secured to the top of the screw. This, of course, is only done when the rod has been passed into the cradle. The result in action causes the cap to be brought against the top of the screw, and more specifically, secure the rod within the cradle.

In the preferred embodiment, the head of the primary screw is slightly ovoid rather than perfectly round. There is located within the top of the screw a bar that is horizontally oriented or, more specifically, oriented along the widest dimension of the ovoid shape. In the preferred embodiment, when the screw is passed into position, the screw is oriented so that the widest dimension of the ovoid shape of the head is perpendicular to the orientation of the connecting rod when in position. Thus, in this embodiment, when the screw is placed into the pedicle it is secured down until the screwhead is oriented in this fashion.

Furthermore, the horizontal bar is preferably located on the side of the screwhead closest to the receiving screw. When the connecting rod is passed into position, the cap is brought down against the head of the screw using the ratcheting mechanism to secure the cap into position. After the cap has been secured into position, the horizontal bar is deployed as a locking mechanism. In this fashion, it is noted that the horizontal bar is fitted within a tract which is located within the walls of the screwhead. This tract is essentially a slight indentation into which the rounded ends of the horizontal bar fit. When the bar is deployed, the bar is rotated around the long axis of the screw. In this fashion, it is rotated from being perpendicular to the orientation of the connecting rod to being merely parallel to the connecting rod. Because of the ovoid shape of the screw, the horizontal bar is longer than the shortest dimension of the ovoid. Hence, when it is rotated around the long axis of the screw, it reaches a point where it becomes locked against the walls of the screwhead. In this fashion, it serves to secure the cap in place, and hence serves as a locking mechanism.

Alternative embodiments are also considered. In one alternative embodiment a side of the screwhead may be placed on a slidable tract with a series of pins or piston rods, again employing the same ratcheting mechanism described above. In this embodiment, the cradle into which the connecting rod fits is actually enlarged in the undeployed position. After the connecting rod is passed into position, the slidable side is then brought against the rod and the immobile side of the screwhead and again secured into position with the use of the ratcheting mechanism as applied to a plurality of pins that are located either above and/or below the connecting rod. In this embodiment, a device is used to draw the two sides of the screwhead together thus ratcheting the mechanism closed and securing the rod into place.

In yet another alternative embodiment, two components of the screwhead can be connected by a hinge and can be closed by rotation around that hinge. This can be done either by rotating a deployable component which is positioned vertically above the nondeployable component, or the deployable component can be positioned below, and to the side of the nondeployable component. In either of these embodiments, the deployable component is rotated around the hinge and ultimately locked together using the ratcheting mechanism. When this is done, in the fully deployed position, these two components create a cradle into which the rod is secured.

It is still another object of this invention to provide an alternative embodiments by which a steerable device can be navigated into this plane to position bone graft in the plane between the transverse processes or sacral ala being fused.

It is yet another object of this invention to initially contemplate a series of uniquely designed bone graft packets that will facilitate placement onto the plane between the transverse processes or sacral ala being fused. It is even still a further object of this invention to provide as the preferred embodiment a system of cannulas and trocars which may be introduced from an independent position superior and inferior to the areas of fusion.

It is yet an even further still object of this invention to provide for a system through which the cannula-trocar complex can serve as a guide for an osteotomy instrument to accomplish the necessary osteotomy of the transverse process or sacral ala.

It is an even further object of this invention to provide an alternative embodiment which provides a series of methods and devices that will facilitate injection of demineralized bone graft, bone gel, methylmethacrylate, or any other injectable substance deemed appropriate by the operating surgeon under the clinical circumstances of the individual case into the plane between the transverse processes or sacral ala.

It is even an additional object of this invention to provide these systems and devices in a fashion that will avoid injury to tissues not otherwise involved in the surgical procedure.

The above and other objects of this invention may be achieved by the use of a system and devices that are introduced into the procedure after the pedicle is initially entered into by a tap or probe. The shaft of the tap or probe will serve as a guide for an osteotomy tool including a shaft, the inner diameter of which fits around the outer diameter of the shaft of the tap or probe. Alternatively, the tool may attach onto the lateral aspect of the shaft of the tap or probe or, as yet another alternative embodiment, may be a component of the working channel. Regardless of the embodiment, the device includes a footplate that presents along its leading edge an osteotomy device with either a wire bone-cutting device or an oscillating saw that initially engages the cortex of the dorsal aspect of the transverse processes or sacral ala along the lateral aspect of the entry into the pedicle.

With a wire osteotome, the wire is rotated by a hand-driven shaft controlled by the surgeon that activates by a thumbscrew or dial on the trailing end of the device. This trailing end protrudes from the patent's body so that it is controlled by the surgeon's hand. The turning of the thumbscrew, dial, ball or any other embodiment of a hand-driven mechanism will transfer the rotating vector down the shaft and activate a series of one or more gears which will drive the wire osteotome. Alternatively, a similar mechanism of a hand-driven drill shaft will activate an oscillating saw.

### BRIEF DESCRIPTION OF THE DRAWINGS

FIG. 1 shows an apparatus for performing surgical procedures on a spine in accordance with an embodiment of the present invention;

FIG. 2 shows a working channel set on a pedicle in accordance with an embodiment of the present invention;

FIG. 3 shows a vertebrae with screws and a connecting rod set in the vertebrae in accordance with an embodiment of the present invention;

FIG. 4 shows a pedicle screw in accordance with an embodiment of the present invention; and,

FIG. 5 shows an osteotomy instrument in accordance with an embodiment of the present invention.

### DETAILED DESCRIPTION

As is typically done with surgical procedures involving the spine, the patient is first put in the prone position. The entry point to the base of the pedicle is identified using standard, well-known radiological landmarks. Traditionally, if a somewhat oblique radiological projection is utilized, the base of the pedicle can be seen as a somewhat obliquely-oriented oval at the junction of the transverse process and the facet joint. A skin incision is made above the identified pedicle, and a trocar 1 passed through.

In a preferred embodiment, the trocar has a radiolucent shaft 2 and a leading end 3 that is radioopaque. Furthermore, the leading end 3 features either a sharp tip, a knurled tip, or a tip that can, in some other fashion, engage the cortical bone overlying the entry to the pedicle. The trocar 1 is passed through the incision and into the base of the pedicle, using radiological guidance, consistent with the method previously described by the inventors. Once the tip of the trocar pierces the base of the pedicle, a series of dilators 5 are then passed over the trocar 1 to prepare the tract for a working channel 6 that will be passed over the outermost dilator.

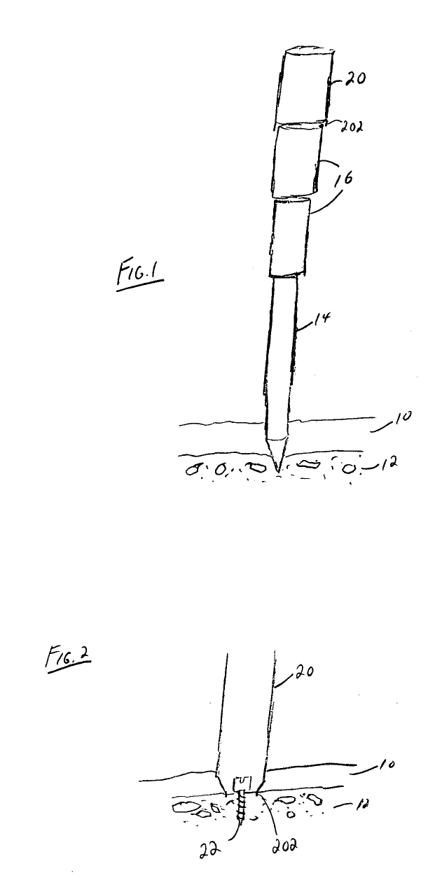

FIG. 1 shows a trocar 14 piercing a skin and muscle layer 10 and setting into a pedicle 12. A series of dilators 16 are placed over the trocar 14 to expand the incision and opening in the skin 10. The working channel 20 is the outermost dilator.

The leading end of the working channel 20 has prongs 202, that may be retractable, to stabilize the channel 20 to the pedicle 12. These prongs may be driven into the bone manually or with a mallet or slap-hammer. Once the working channel 20 is set, the interior dilators may be removed.

A drill (not shown) may be introduced through the working channel to further create a tract for the pedicle screw. A drill-tap combination, or a drill followed by a tap may alternatively be employed. As a further alternative, a self-tapping screw may be utilized.

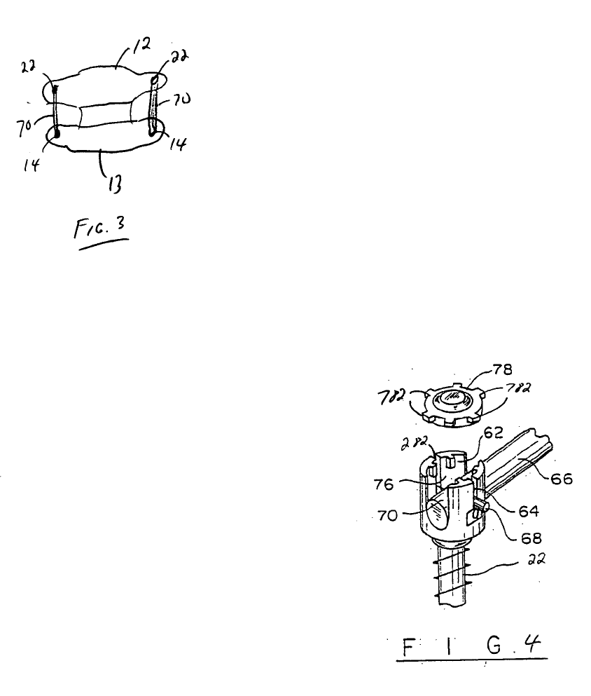

Referring to FIG. 2, a primary screw 22 is set into the pedicles of a vertebra 12via the working channel 20. The entire procedure is repeated to place a secondary, or receiving screw 14 in the pedicle of an adjacent vertebra 13. The primary 22 and secondary 14 screw heads are uniquely configured to facilitate passage and setting of a connecting rod between the two, as shown in FIG. 3 which is a posterior view of adjacent vertebrae 12 and 13. Primary screws 22 are set into the pedicles of the top vertebra 12, secondary screws 14 are set into the bottom vertebra 13, and the connecting rod 70 is set into the screw heads.

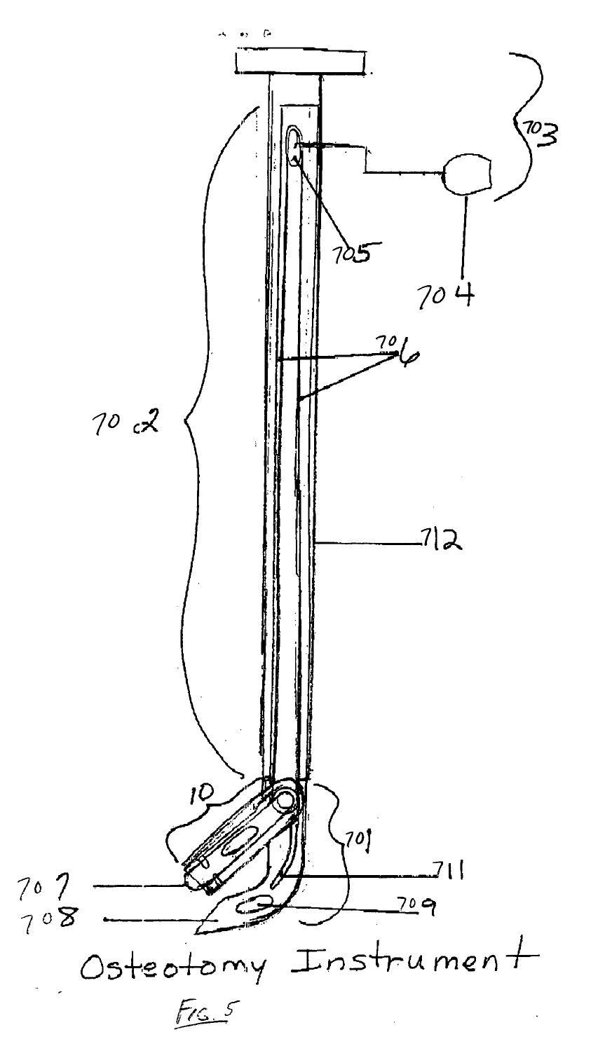

Referring to FIG. 4, the cradle of the screw head 76 that ultimately houses the connecting rod is inclined slightly towards its floor to the receiving screw. This incline in the floor of the cradle pushes the connecting rod towards the secondary screw when it is inserted. In addition, a horizontal bar is positioned on the primary screw head that passes from one wall of the cradle to the other, on the side of the cradle closest to the receiving screw. This bar is designed to ensure that the connecting rod 70 is directed towards the receiving screw head.

A mechanism housed in the leading end of the shaft of the working channel fits into the primary screw, and allows for the connecting rod to be driven into the tissues intervening between adjacent screw heads. The mechanism is preferably a gear that engages teeth on the rod to push the rod downward, through the intervening tissue and into the primary and secondary screw heads. A trocar may be passed through the intervening tissue first to clear the area for the connecting rod.

In a second alternative embodiment, two components of the screw head can be connected by a hinge and can be closed by rotation around the hinge, like two horizontal doors opening up vertically. The two components may be on top of each other or side-by-side. When this is done, in the fully deployed position, these two components create a cradle into which the rod is secured.

The head of the primary screw has a cap 78 positioned directly above the cradle of the screw. Furthermore, a plurality of pins 782 extend from the undersurface of the cap into channels 282 in the walls of the screw head. The channel walls constitute the sides of the cradle. The pins 782 have a swelling at their leading end. The channels 282 have a series of narrowings within them that engage the pins 782.

The length of the channels 282 is such that when the pins 782 are fully inserted into the channels, the cap is securely fitted to the top of the screw. As the leading end of the pins passes through the series of narrowings, the leading end of the pins is caught in an irreversible fashion. Thus, as the cap and its attendant pins are pushed into the top of the screw, the pins are sequentially captured by the narrowings and the cap becomes secured to the top of the screw.

As a result, the cap is brought against the top of the screw, and secures the rod within the cradle. In the preferred embodiment, the head of the primary screw is slightly ovoid rather than perfectly round. There is located within the top of the screw a bar that is horizontally oriented or, more specifically, oriented along the widest dimension of the ovoid shape.

In use, the screw is set so that the widest dimension of the ovoid shape of the head is perpendicular to the orientation of the connecting rod when in position. Thus, when setting the screw into the pedicle, it is turned until the screwhead is oriented in this fashion. Furthermore, the horizontal bar shall be on the side of the screwhead closest to the receiving screw.

After the primary and secondary screws are placed in their respective pedicles, a tract between the screw heads passing through the intervening soft tissues is created.

A series of downward-spiraling tracts direct the horizontal bar into a locking position with a twisting motion by setting the ends of the bar into the tracts. To lock the bar into position, it is rotated around the long axis of the screw, in the horizontal plane. In this fashion, it is rotated from being perpendicular to the orientation of the connecting rod to being parallel to the connecting rod.

Because of the ovoid shape of the screw head, the horizontal bar is longer than the shortest dimension of the ovoid. Hence, when it is rotated around the long axis of the screw, it reaches a point where it becomes locked against the walls of the screwhead. In this fashion, it serves to secure the cap in place, and hence serves as a locking mechanism.

In accordance with a first alternative embodiment of the present invention, the pins of the cap may engage a series of downward-spiraling tracts in the walls of the screw head to lock the cap in position without the need for a nut or bolt.

In accordance with a second alternative embodiment of the present invention, the screw head may include two halves, one fixed and the other laterally slidable towards and away from the fixed half. After the connecting rod is passed into position, the slidable half is brought against the fixed half and again secured into position with the use of the ratcheting mechanism as applied to a plurality of pins that are located either above and/or below the connecting rod. A device is used to draw the two sides of the screwhead together thus ratcheting the mechanism closed and securing the rod into place.

The tissue intervening between the screw heads can be cleared in preparation for passage of the connecting rod, and the working channel serves as a guide for the placement tool.

A flexible locator device can be passed through the working channel and into the head of the receiving screw, with this flexible locator then serving to guide the placement tool to its proper position.

In a preferred embodiment, the connecting rod is configured such that the leading end is sharply tapered, thus allowing the rod itself to act as a trocar and bluntly dissect the soft tissues in its passage towards the secondary, or receiving screw. In this embodiment, this passage may require insertion and retraction of the rod into the tissues, repeating this action several times; in each instance accomplishing further dissection of the intervening soft tissues prior to completion of the passage from the screw head of the primary screw to the screw head of the receiving screw. Once the connecting rod has been successfully passed, the rod is secured in place with locking screws.

In an alternative embodiment, a device for formally passing a rod trocar through the soft tissues in preparation for passage of the connecting rod is utilized. Specifically, after the placement of a primary screw, as described above, a guide is used to pass a trocar housing device onto the head of the primary screw. This guide can take a number of forms.

In a preferred embodiment, after the primary screw is set, the screwdriver, used to set the screw, is maintained in contact with the primary screw head. The working channel is then withdrawn, and the trocar-housing device is passed over the screwdriver while the screwdriver remains in place. The screwdriver is then withdrawn. The trocar-housing unit is composed of a leading end, a shaft, and a trailing end. The leading end houses the rod trocar proper, and engages the screwhead of the primary screw, while the trailing end is the component of the trocar housing device with which the surgeon interfaces, and allows the surgeon to deploy the rod trocar. The shaft is used to pass the device into position.

In general, it is anticipated that the rod trocar is housed within the leading end of the trocar-housing device, located on the side of the leading end of the device that is furthest from the receiving screw. It is further anticipated that the rod trocar is deployed by activating the trailing end of the device, and that this activation, in turn, causes a piston rod that is incorporated within the wall of the shaft of the trocar housing device to be displaced. This displacement may be the result of pushing on the trailing end of the piston rod, activation of a lever, screwing downward causing displacement of the piston rod, or any other mechanism that will induce displacement of the piston rod. This displacement, in turn, causes the trocar to be deployed.

The rod trocar is of the same diameter, or slightly larger, as any of the connecting rods to be utilized, and has a leading end and a trailing end. It also is curved in the same angle and to the same extent. The trailing end interfaces with the leading end of the piston rod in such a fashion that displacement of the rod will cause deployment of the trocar. The leading end of the trocar comes to a point and is sharpened sufficiently so that when the rod trocar is deployed, it will easily pass through the soft tissues that are found between the screwheads of the two screws.

When the device is in proper position, the surgeon deploys the trocar. This is accomplished in accordance with the mechanism utilized to deploy the trocar. The trocar is deployed as the result of the interaction between the trailing end of the trocar and the leading end of piston rod. The trocar is, therefore, advanced through the soft tissues lying between the screwhead of the primary screw and the screwhead of the receiving screw. This action creates a tract in the soft tissues, and this tract can be easily negotiated by the connecting rod.

In an alternative embodiment, a tract in the soft tissues found between the two screwheads is created by a flexible cannula, the leading end of the flexible cannula which is outfitted with either an electrocautery lead, ultrasonic desiccators, or LASER-assisted power source. In any of these embodiments, the cannula is passed down through the lumen of the working channel until the leading end of the cannula is in contact with the cradle of the screwhead. At that point, the slight incline of the floor of the cradle will induce the flexible cannula to be directed through the space created by the horizontal bar and into the intervening soft tissues. At that point the power source of the leading end of the cannula is activated, and the tract through these soft tissues is created.

In any event, once this space is opened by one of the methods described above, the connecting rod is inserted between the screw heads. In a preferred embodiment above, the connecting rod is tapered much like the rod trocar, and passage of a trocar is not necessary. In this embodiment, the lumen of the working channel is somewhat irregular. This is specifically so that it may accommodate both the primary screw and the connecting rod.

After the screw is set, and a screw has been placed into the pedicle of the adjacent vertebra, then the connecting rod is passed down the lumen of the working channel. When the rod is in its initial position, it is found in the leading end of the working channel, with the curve of the rod concave toward the adjacent screw. The rod is initially passed down the lumen of the working channel with a primary rod pusher. As it is positioned in the leading end of the working channel, it would not be able to be negotiated.

At this point, a panel on the wall of the working channel, which would be located at a point on the wall opposite the anticipated course of the connecting rod, opens. This allows for the connecting rod to be repositioned such that it is in ideal position to negotiate the space between the two screwheads. The trailing end of the connecting rod falls through the space created by the opening of the panel. Then, the final rod pusher is passed down along the outside of the working channel. The leading end of this device then captures the trailing end of the connecting rod, and the final rod pusher will then encourage the connecting rod into position. The locking nuts are then passed down, and the procedure is completed.

In accordance with further aspects of a preferred embodiment, a rod insertion device has a unique configuration of the leading end, which is slightly curved to recapitulate the curve of the rod. The rod is loaded into the leading end prior to placement of the device into the patient's body. After the primary screw is set, a guide brings the insertion device to its proper position.

A flexible guide shaft consisting of a leading end, a shaft, and a trailing end is passed down through the working channel 6. The leading end of the guide will, in some fashion, interface with the screwhead of the primary screw. The working channel is then removed.

In this way, the guide facilitates insertion of the device into the screw head. Further, the chance of surrounding tissue insinuating itself into the screw head is reduced. The flexibility of this guide allows the curved leading end of the insertion device to negotiate over the flexible guide. The device is then passed down into its proper position being guided by the flexible guide and the horizontal bar of the primary screw. The flexible guide is passed into a lumen in the insertion device. The insertion device should be positioned such that the curvature of the leading end is continuous with the slight incline of the cradle in the primary screwhead. Once in position, the insertion device is used to insert the connecting rod by initially removing the flexible guide. The connecting rod is in place in the leading end of the device, with a rod pusher also already in place.

The rod pusher consists of a leading end, a shaft, and a trailing end. The leading end is unique in its interface with the connecting rod. Specifically, the leading end is flexible to follow the curvature of the rod. To provide the requisite flexibility, the leading end may be constructed of a flexible substance. Alternatively, the leading end may be a series of linked or unlinked spheres, balls, or wheels that can follow the curvature of the rod. The trailing end of the rod pusher provides a handle or other means by which the surgeon can interface with this component of the rod insertion device. By interacting with the trailing end of the rod pusher, the surgeon will accomplish displacement of the rod pusher and the connecting rod advances into the final position. This is the result of the curvature of the insertion device, in combination with the geometry of the cradle of the primary screw.

In accordance with further aspects of an alternative embodiment, the leading end of the working channel has a detachable base. After setting the primary screw, the working channel is removed and the base is left surrounding the screwhead. Prongs or locator pins extend upwardly from the base, which can be of any length, for guiding the insertion device into position. The connecting rod is then inserted as previously provided.

Once the screws and connecting rod are set, thereby stabilizing the vertebrae, the necessary surgical procedure can be performed. The stabilization tools and method disclosed herein may be used with any surgical procedure that requires stabilization of vertebrae. Typically, some bone growth material is implanted into the affected area.

In accordance with further aspects of the present invention, a steerable osteotomy device is provided that can be navigated into the necessary plane to obtain the bone graft between the transverse processes or sacral ala being fused.

The bone graft packets are designed specifically to facilitate placement onto the plane between the transverse processes or sacral ala being fused. In addition, cannulas and trocars may be introduced from an independent position superior and inferior to the areas of fusion. The cannula-trocar complex may then serve as a guide for the osteotomy instrument to accomplish any necessary osteotomy.

Bone growth materials that may be used in conjunction with the invention include demineralized bone graft, bone gel, methylmethacrylate, or any other injectable substance deemed appropriate by the operating surgeon under the clinical circumstances of the individual case.

Referring to FIG. 5, the osteotomy tool is composed of a footplate 708 that presents along its leading edge an osteotomy tool that may be a wire bone-cutting device or an oscillating saw. The cutting edge 707 device initially engages the cortex of the dorsal aspect of the transverse processes or sacral ala along the lateral aspect of the entry into the pedicle. In a wire osteotome tool embodiment, the wire is rotated by a hand-driven shaft controlled by the surgeon, activated by a hand-driven mechanism 704, such as a thumbscrew or handle, on the trailing end of the device. The trailing end protrudes from the patient's body and is controlled by the surgeon's hand.

The primary embodiment of the second component of this invention involves a mechanism by which the osteotomy in the transverse process or sacral ala can be directed laterally. This will permit the establishment of a coronal osteotomy through the transverse process and is critical in developing a vascularized bone flap to serve as the bed for fusion graft material. In the primary embodiment, the initial osteotomy at the base of the transverse process or sacral ala along its medial aspect is established by the use of the wire osteotome. The osteotome can then be directed laterally with the use of a trocar which is either flexible or articulated. The trocar is directed by the interior of the shaft 702, out through a curved endpoint 709 and directed laterally into the transverse process or sacral ala. As the osteotome-trocar complex is advanced, the coronal osteotomy is completed being carried from the medial aspect of the transverse process or sacral ala to the lateral aspect of the transverse process.

In another embodiment, the osteotomy is created as the result of access to the transverse process or sacral ala via two separate introduction ports. These trocar-cannula complex are introduced at a point lateral to the working channels used to introduce the screws and a point superior to the most superior transverse process or inferior to the most inferior transverse process or sacral ala. The cannula-trocar is then introduced to the point where it rests against the medial aspect of the transverse process and an osteotomy is introduced which creates a coronal osteotomy. The instrument and osteotome can then be gently rotated in such a fashion that the osteotomy is carried laterally and the split-thickness autograft flaps developed. The free space is then developed as further described and packed with bone graft material.

An alternative embodiment of the second component of this device is an elbow-type joint which is located along the shaft at an approximate distance equal to the length of the transverse process or sacral ala. The mechanism by which this device works is that it engages the bone of the posterior cortex of the transverse process or sacral ala lateral to the entry into the pedicle. This should avoid injury to the facet artery which can often lead to significant hemorrhage. The transverse process is initially entered into in a direct downward approach and then as the shaft of this device is gently inserted into the body of the patient, the elbow joint engages so that the rotating or oscillating osteotome is brought from a vertical plane to a horizontal plane along the coronal plane of the transverse process or sacral ala. It is contemplated that two or more of these devices may be used in series which each footplate being somewhat longer than the previous. In this fashion, the posterior aspect of the transverse process or sacral ala is divided or osteotomized in the coronal plane and can be elevated dorsally while still attached to muscle which carries nutrient arteries to the periosteum. The creates a vascularized bone flap to be utilized in the graft. This procedure is performed bilaterally at each level to be incorporated into the fusion.

After the vascularized bone flaps have been raised at each level to be included in the fusion, the next issue to be dealt with is the musculotendinous and other soft tissue components which is positioned between and occupies the space between the transverse processes or sacral ala—commonly referred to as the “lateral gutter”, in spinal surgical parlance. This step is actually key to the maturation of a bony union or fusion, as this is the region which must be “bridged”, in order to accomplish the fusion of the two or more adjacent vertebrae in this particular type of fusion. It is postulated that without creating an adequate free space within these soft tissue planes, bone graft material presented to be incorporated into the fusion will be reabsorbed rather than achieving maturation into a satisfactory bony union or bridge.

Since excessive soft tissue resulting from inadequate clearing and creation of a free space may result in soft tissue insinuating itself between the elements of bone graft material which has been positioned into place. The ability of the individual bone elements to create a union with other bone elements is reduced, and also increases the rate of resorption of bony elements lying within the tissue planes. Resorption, or reabsorption of the bony graft material placed to create a fusion, is a ubiquitous phenomenon, which occurs to some degree, in virtually every case of attempted fusion.

It is often thought that the success of a fusion can be euphemistically reduced to a “race”, between the time that the bony elements begin to create osseous or bony bridges between each other, and the time that the recipient's body will physiologically reabsorb the graft. Once osseous material begins to bridge the gap between the individual body elements, it is thought that generally, the patient will successfully mature the fusion bed.

On the other hand, if enough graft material is reabsorbed prior to the foundation of an adequate network of osseous connections, then a failure to fuse (referred to as ‘pseudoarthrosis’) is thought to occur. One of the principle values of pedicle screw fixation is that by stabilizing the spinal elements to be fused, a favorable environment for the formation of these primitive osseous connections so vital to the evolution and maturation of a successful fusion.

There are several embodiments of the set of devices and methods utilized to create a free space within the tissue planes of the lateral gutter.

In a first embodiment, a series of soft-tissue cutting tomes are utilized to create a tract through the soft tissues between the heads of the screws, into which the screws can then be passed and rotated into place. As an augmentation to that embodiment, the present invention provides for a cutting tome which can be directed laterally into the soft tissues from the base of a shaft that can be anchored in the screwhead. This will separate the tissue planes of the soft tissues lying between the transverse processes or sacral ala, along a coronal plane, creating a free space into which the proposed bone graft material can be juxtaposed.

An alternative embodiment provided is the use of flexible probes which can be passed down through a side port of the working channel, or along a channel connected to the long axis of the shaft of the tap, or introduced into the operative field by whatever means the osteotomy device is introduced. A variety of functional tips applying either mechanical, pneumatic, thermal, electrical, laser or chemical dissection techniques are then utilized to dissect the soft tissues and create a free space into which the bone graft can then be juxtaposed. After the free space is created, a flexible instrument which has as a leading edge a pincer or grasping component is introduced. This instrument will then guide into position a uniquely designed bone delivery system composed of multiple components or elements which contain morselized bone within a gel or capsular matrix.

An alternative embodiment is provided in which a separate entry site is utilized to introduce the set of devices that are provided to create a free space between the tissue planes of soft tissues interposed between the transverse processes or sacral ala of the vertebrae being considered for fusion. In this embodiment, the transverse processes or sacral ala are again divided in the coronal plane, creating vascularized bone flaps that contribute autograft to the proposed site of fusion.

After this step, introduction sites are selected and ports in the form of a trocar-cannula system are introduced into the field from a point superior to the cranial end of the proposed fusion and inferior to the caudal end. These ports are introduced through the skin utilizing a sharpened trocar, or with any other introduction system. The port is then passed to a point which represents the junction of the transverse process or sacral ala with the entry into the pedicle.

Thus, as the transverse process or sacral ala has already been divided in the coronal plane, the port is hence introduced into an area between the two vascularized bone flaps. The trocar is then removed, and a probe with a uniquely-designed expandable, flexible tip is introduced through both ports. As these probes advance through the soft tissues, the free space, which is necessary for the placement of the bone graft so that it is in juxtaposition between the two vascularized bone flaps, is created.

A further description of the uniquely designed tip is given below, but a further description is helpful at this time. The tip of the probe contemplated herein is comprised of multiple flanges, flaps or petals, and can be round, ovoid, tulip-shaped, or any other configuration which can match and be negotiated through the introduction port. Furthermore, a shape such as that discussed above would better facilitate allowing the probe to insinuate itself into the soft tissues. It can have a smooth leading edge, but it would be more favorable to have a pointed or sharp tip as the leading edge, again to facilitate entry into the soft tissues. The trailing edge is connected to the operating mechanism and is the means by which the surgeon is able to direct the orientation of the probe as well as control the opening and closing of the flanges, flaps, or any other configurations utilized in this application. As the probe is advanced, the opening and closing of the flanges, along with rotating and redirecting the probe, creates the tissue plane necessary for the placement of the bone graft material.

Serving as an extension of this concept, there is contemplated yet another alternative embodiment. In the alternative embodiment provided in the paragraph above, a mechanical method for creating the necessary tissue plane and free space is disclosed. The unique, novel and non-obvious component of this invention includes that a power-generated lesion system, including laser, ultrasonic cavitation, adaptation of harmonic scalpel technology, or any form of electric current (AC or DC) could be adopted, and may be considered desirable for this technique. It can be postulated that in this embodiment, after the cannulated probes are passed into position in the tissue plane between the vascularized flaps of the transverse processes or sacral ala that have been split by the osteotomies, the trocars of both the superior and inferior ports are removed. Then, a probe connected to the power source, be it laser, ultrasonic, electrical or any other power source is positioned through the ports. The probes are then utilized to create the necessary free space for positioning the bone graft material.

Once the tissue planes have been properly separated and the free space to accommodate the graft material is created, the methods disclosed herein provide for a primary method as well as alternative embodiments to introduce bone graft material into the free space created to accommodate the bone graft material so as to achieve juxtaposition between the graft material and the vascularized autologous flaps.

In the primary method, the bone graft material is represented by a unique non-obvious and novel bone delivery system which is represented by multiple chambers, components, or elements. These contain within them bone graft material, either in the form of morselized bone, demineralized bone, partially demineralized bone, all of which may or may not be suspended within a gelatinous matrix. Furthermore, the component chamber or element is encased within a rapidly dissolvable encapsulating substance. One or more of these chambers are then connected with dissolvable connection material such as suture or other flexible, dissolvable material.

In the preceding specification, the invention has been described with reference to specific exemplary embodiments thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1. An osteotome device comprising:
a shaft having a first end and a second end;
a handle on the first end;
a cutting tip on the second end;
a chain connecting the handle and the tip so that manipulation of the handle actuates a cutting motion of the cutting tip.
